SEQUENCE LISTING (3 sequences)

SEQ ID NO: 1 - DNA, 24 bp, synthetic construct
  source 1..24: mol_type = other DNA
  misc_feature 1..24: note = PCR_primers_fwd_seq
atgttggtgtatcttgagatcctc

SEQ ID NO: 2 - DNA, 21 bp, synthetic construct
  source 1..21: mol_type = other DNA
  misc_feature 1..21: note = PCR_primers_rev_seq
tccccagttgtactccatctg

SEQ ID NO: 3 - DNA, 3001 bp, Homo sapiens
  source 1..3001: mol_type = genomic DNA
  ncRNA 1..3001: ncRNA_class = lncRNA
cttgaaacaaaatactggtaaagaaaaagctatctattctagtggatagaaatctggagaaatatcacaaatgttctgagccccagacctactgagacttttgcaggaagcattacctctatcctacctggcagacaaaacactgcctgcccttttttgctagataattctaagtgaggaagcctgtggacttactaaatgacctatatgatctgcgaggagtaaaacacaaccattctgaagggaaacatacccggaaccataagagtctttcactaggaccagggtaagatggtttggggggaactgtcaaatatttatggctttttgttcttacactgaataagtagaatttttaaaaaatgtctggtggtgaaaagaacttaaaacagttgaagtattcggggtgtctaaagtaaattaaaacaggctgggtgcatcataatttagttagccagttctttatagatagacatttagtttatttctagctcttttctattttttggcatgcaagcatcattgtacatgtatctcatcacatttatttgattatactcttatgatcattgccttaaaatatagttactaagagtttgtaaaacttccaaacttttgatacatattgttaagttgccctctaaaaatataataaaagttgccatttccactaaaagtatatgagaatagttgttttcttaatactgtcaccaatattatgtattaccatttattaaaaatattttttaaaacttgtctaatctgaaatgtgaaaaatggtacattgttttaagattacttgtgagaacaatgatcataatcattatcattttttgcgggagtatcttactttcctctcaaaatagttcaatgagtaggtcctattaattcatatattcattggctatttctgccttctaaaaattaatttgtgtgtatgtcttttgtgtgtttttctattaggatttttgtttttttatttttatttttatttttatttttttttgagacggagtctcgctctgtcgctcaggctggagtgcagtggcgcgatctcggctcactgcaagctccgcctcccgggttcacaccattctcctgcctcagcctcctgagtagctgggactacaggtgcccaccaccacgcctggctaattttttgtatttttagtagagacggggtttcacggtgttagccaggatggtctcgatctcctgaactcgtgatccgcctgccttggcctcccaaagtgctgggattacaggcgtcagccaccgcgcccggactctattagtatttttttttttttagtgtagaagtgctccttaaagattaaggaaatcaagtttctcctgataatctttttttgggatgtgtctttagcggtacttctgttgatcaaaagtaactgataactcagaaagtatggagaaagtcctaaaattgaacacaaataaaaacaaatgaacctcaatgtgtataaaaatgttaacataaccacacagaataaaaaaaattaatacaagagacttttgaatatattactttgatgtacactctcagggagatacattccaaggacaagaagtactatgaagaaatactaaacattacttagtaggtttcttgttagtagaagtattgaagtattgatagagtaattttgaagttatttgctcaattttagaatgttagaattgagatagatagataggttgattgatattgctgggaaaaagactcttactgaggcataaaggtggtacaaacatgaaatggcacaaggaaaggaagaaccctttggtgtttgattggcactggaaagtatgaacttataattttatatatattggatttgtttctcttagcaacgtctggatttgtaaacacgctccaacaagaagcaggctgcacaagctttgtagatcacaaggagcattaactctcccatgaccttcagatgtggccataggggctgatggacaaagaattaccccctagagctaggagcaatggataccagggagttttccatagaaagctgaaccagagacttgctccataaccaggacagggatcttcacaatctctgaccagaaggatttgcttaccactgtggaaacaatgactgtcatgtttcttctcttctttttttggtgccaaatctatcaccattggcggagaaaaagaaccttctcttagactgtaggtgatctgaccccaggagccacacttggagataactacccatcaccaagagatcctggaatttcaacctgatgatataactggatgggactttgggagtatctcctgtgggaaaaaggaaagaatttcgtttggcaggtcaagtgtagttggatatttatgggtggctaaaggggcagtctgtggaagagacttcttgttatttcttcccaacatccattcctttcttctcctattatgagagatattttgaatttttgacaaatcccagtctcccttgcatctagatgtggccatgagactaagttttggccaaaggggtataaatgatattgttgtacacaactttaggcttgtgaccttaaagtgaaacaggtgtgccctacactgcccattttttcgaccttgttttaactgtggatgatgtggatgaggtggtgagctactatggaccatgagaatcagtggagcatactagagatggtgcaacaataagagaaggagcctgggtccctgaatgacctcatggggtagatgtcctaagctagtatcacactatttactcccagactggtggagaagagagaaataatttctggcgaattcaaaacactgttattttggagccctgttacatgcaccttgacctatattccaaattagataagtaagataggtaagatatcttaatgcactttggcatgctatcattcccaaatacatgggaattaaatttttttggtataaatttacactgagaaacaaaagtatctaaaactaaaagtattattt

(19) United States
(12) Patent Application Publication
(10) Pub. No.: US 2025/0243454 A1
(43) Pub. Date: Jul. 31, 2025

(54) METHODS FOR FACILITATING THE DIFFERENTIATION OF INDUCED PLURIPOTENT STEM CELLS INTO RETINAL GANGLION CELLS

(71) Applicants: Centre for Eye and Vision Research Limited, Hong Kong (HK); The Hong Kong Polytechnic University, Hong Kong (HK)

(72) Inventors: Chien-Ling HUANG, Hong Kong (HK); Shea Ping YIP, Hong Kong (HK); Ka-Hing WONG, Hong Kong (HK); Nonthaphat WONG, Hong Kong (HK); Jiahong SUN, Hong Kong (HK); Zhiwei JIANG, Hong Kong (HK)

(21) Appl. No.: 19/036,052

(22) Filed: Jan. 24, 2025

Related U.S. Application Data
(60) Provisional application No. 63/624,790, filed on Jan. 25, 2024.

(51) Int. Cl. C12N 5/079; C12N 9/22; C12N 15/11; C12N 15/90; C12Q 1/6874; C12Q 1/6881

(52) U.S. Cl. CPC C12N 5/0618; C12N 9/22; C12N 15/11; C12N 15/907; C12Q 1/6874; C12Q 1/6881; C12N 2310/20; C12N 2506/45

(57) ABSTRACT

A method for differentiating induced pluripotent stem cells (iPSCs) into retinal ganglion cells (RGCs) involves deleting a sequence encoding LNC000093 long non-coding RNA from the iPSC genome, culturing the modified iPSCs to generate colonies, and dissociating these colonies to form embryoid bodies. The embryoid bodies are transferred to a first plate to induce neural rosette formation. The neural rosettes are detached and cultured in suspension to develop into neurospheres, which are subsequently transferred to a second plate to promote differentiation into RGCs.

### CROSS-REFERENCE TO RELATED APPLICATIONS

The present application claims priority from U.S. provisional patent application Ser. No. 63/624,790 filed Jan. 25, 2024, and the disclosure of which is incorporated herein by reference in its entirety.

### REFERENCE TO SEQUENCE DISCLOSURE

The sequence listing file under the file name “P3068US01_Seq listing.xml” submitted in ST.26 XML file format with a file size of 7.76 KB created on Jan. 17, 2025 and filed on Jan. 24, 2025 is incorporated herein by reference.

### FIELD OF THE INVENTION

The present invention generally relates to the molecular cell biology fields. More specifically the present invention relates to method for enhancing the differentiation of induced pluripotent stem cells (iPSCs) into retinal ganglion cells (RGCs) through the strategic application of CRISPR editing.

### BACKGROUND OF THE INVENTION

Retinal ganglion cells (RGCs) play a critical role in vision by transmitting visual information from the retina to the brain. Damage or degeneration of RGCs is a hallmark of various retinal diseases, including glaucoma and optic neuropathy. Enhancing the differentiation of induced pluripotent stem cells (iPSCs) into RGCs represents a promising avenue for treating retinal disorders and advancing regenerative medicine.

iPSCs are stem cells reprogrammed from adult cells, such as skin cells, to a pluripotent state, enabling their differentiation into multiple cell types, including RGCs. This capability positions iPSCs as a valuable resource for developing regenerative therapies for retinal diseases.

Despite their potential, achieving efficient and functionally mature differentiation of iPSCs into RGCs remains a significant challenge. Mimicking the intricate developmental processes of retinal development in vitro requires precise replication of signaling cues, growth factors, and small molecules to create an environment conducive to RGC maturation.

Existing protocols for RGC differentiation have attempted to address these challenges. One such protocol involves gene manipulation through lentiviral transduction, specifically overexpressing NGN2, a key transcription factor within the basic-helix-loop-helix (bHLH) network. In this approach, lentiviral transduction is performed on day −1, followed by doxycycline-induced NGN2 expression on day 0. On day 2, glial cell-derived neurotrophic factor is introduced, and cells are transitioned to RGC SATO growth medium with the Notch inhibitor DAPT for the next four days. While innovative, this method is constrained by safety concerns, scalability issues, and potential off-target effects associated with lentiviral vectors.

Current RGC differentiation methodologies face additional challenges, including limited efficiency, precision, and real-time monitoring of cellular transitions during the differentiation process. The reliance on endpoint analyses fails to provide dynamic insights, and the predominant use of genetic manipulation raises concerns about genome integrity and safety for clinical applications.

The present invention addresses these limitations by introducing a novel approach to enhance RGC differentiation from iPSCs, emphasizing safety, scalability, and real-time cellular analysis.

### SUMMARY OF THE INVENTION

It is an objective of the present invention to provide materials, or methods to solve the aforementioned technical problems.

In accordance with a first aspect of the present invention, a method for facilitating the differentiation of iPSCs into RGCs is provided. Particularly, the method includes the following steps:
deleting a sequence encoding a long non-coding RNA, LNC000093, having a sequence of SEQ ID NO: 03 from the genome of iPSCs;
culturing the LNC000093-knocked-out iPSCs to generate iPSC colonies;
dissociating the iPSC colonies to form embryoid bodies;
transferring the embryoid bodies to a first plate to induce the formation of neural rosettes on the gelatin-coated first plate;
detaching the neural rosettes from the first plate and culturing the detached neural rosettes in suspension to form neurospheres; and
transferring the neurospheres to a second plate to facilitate the differentiation into RGCs.

In accordance with one embodiment of the present invention, the first plate is a gelatin-coated plate supplemented with 10% fetal bovine serum (FBS) to enhance neural rosette formation.

In accordance with one embodiment of the present invention, the detached neural rosettes are cultured in suspension in hES medium comprising 10% FBS and 10 μM Notch inhibitor DAPT for a period of 5 to 7 days to form the neurospheres.

In accordance with one embodiment of the present invention, the formation of the embryoid bodies comprises culturing the iPSC colonies in a human embryonic stem (hES) medium without basic fibroblast growth factor (bFGF).

In accordance with one embodiment of the present invention, the method further includes a step of maintaining the second plate with a medium supplemented with fresh Notch inhibitor DAPT, replenished every two days, to support RGC differentiation.

In accordance with one embodiment of the present invention, the method further includes a step of validating the identity of the RGCs by assessing the expression of retinal ganglion cell-specific markers, including POU4F2, SNCG, and BRN3A, using RT-qPCR or immunostaining techniques.

In accordance with one embodiment of the present invention, the method further includes a step of monitoring the differentiation process through single-cell RNA sequencing to evaluate the transcriptional profiles of intermediate and mature RGCs.

In accordance with one embodiment of the present invention, the method further includes a step of enriching the resulting retinal ganglion cells through magnetic-activated cell sorting (MACS) based on the expression of specific RGC markers.

In accordance with one embodiment of the present invention, the second plate is a laminin-coated plate.

In accordance with a second aspect of the present invention, a method for facilitating retinal organoid differentiation is introduced. Specifically, the method includes the following steps:
culturing the aforementioned LNC000093-knocked-out iPSCs in an Essential 6 medium for approximately 28 days to form neuroepithelial-like structures;
transferring the neuroepithelial-like structures to a suspension culture medium for approximately 7 days to form floating organoids;
plating the floating organoids onto a Matrigel-coated surface and culturing them for approximately 7 days to form retinal organoids; and
harvesting the retinal organoids.

In accordance with one embodiment of the present invention, the Matrigel-coated surface is pre-treated with selenium-nanoparticles (SeNPs).

In accordance with one embodiment of the present invention, the SeNPs are introduced into the culture medium at a concentration of approximately 1 μM prior to harvesting the retinal organoids.

In accordance with one embodiment of the present invention, the Essential 6 medium is replaced with an Essential 6 medium supplemented with 1% N2 supplement after 2 days of initial culturing to enhance differentiation efficiency.

In accordance with one embodiment of the present invention, the suspension culture medium comprises a DMEM/F12 medium supplemented with 1% MEM nonessential amino acids, 2% B27 supplement, and 10 ng/mL FGF2 to support the growth of floating organoids.

In accordance with one embodiment of the present invention, the treatment of SeNP enhances the development and functions of the retinal organoids.

### BRIEF DESCRIPTION OF THE DRAWINGS:

The patent or application file contains at least one drawing executed in color. Copies of this patent or patent application publication with color drawing(s) will be provided by the Office upon request and payment of the necessary fee.

Embodiments of the invention are described in more details hereinafter with reference to the drawings, in which:

FIGS. 1A-1B depict the workflows in according to some embodiments of the present invention, in which FIG. 1A depicts a schematic of the integrated process for facilitating iPSC-RGC differentiation, and FIG. 1B depicts the nanoparticle-embedded retinal organoid culture procedure;

FIGS. 2A-2D depict the validation of LNC000093 CRISPR-deletion effect and iPSC-RGC differentiation, in which FIG. 2A shows the gel electrophoresis results, FIG. 2B depicts the qPCR analysis results, FIG. 2C displays the morphologies during the differentiation of iPSCs into RGCs at different time points, and FIG. 2D depicts the RT-qPCR analysis of a panel of RGC-specific markers;

FIGS. 3A-3G depict the single cell RNA sequencing analysis of iPSC-derived RGCs, in which FIG. 3A depicts the clustering analysis, FIG. 3B shows the expression of RGC markers among the clusters, FIG. 3C depicts the RNA velocity analysis, FIG. 3D demonstrates the percentage of cells in each cluster, FIG. 3E depicts the examination of marker genes related to RGC maturation and axon guidance, FIG. 3F shows the differentially expressed genes (DEGs), and FIG. 3G exhibits the Gene Ontology (GO) analysis results; and

FIGS. 4A-4H depict the single cell RNA sequencing analysis of iPSC-derived retinal organoids, in which FIG. 4A shows the clustering analysis of the retinal organoids, FIG. 4B depicts the DEG analysis of the SeNP-LBP condition and the control organoid, FIG. 4C demonstrates the GO enrichment analysis of the genes related to eye development, visual system development, and sensory system development, FIG. 4D depicts the DEG analysis of major retinal cell types, FIG. 4E GO enrichment analysis revealing the role of SeNP-LBP in regulating mitochondrial activity in RGCs, FIG. 4F demonstrates the DEG analysis in RPCs, FIG. 4G shows the DEG analysis of cones, and FIG. 4H displays the DEG analysis of RPE.

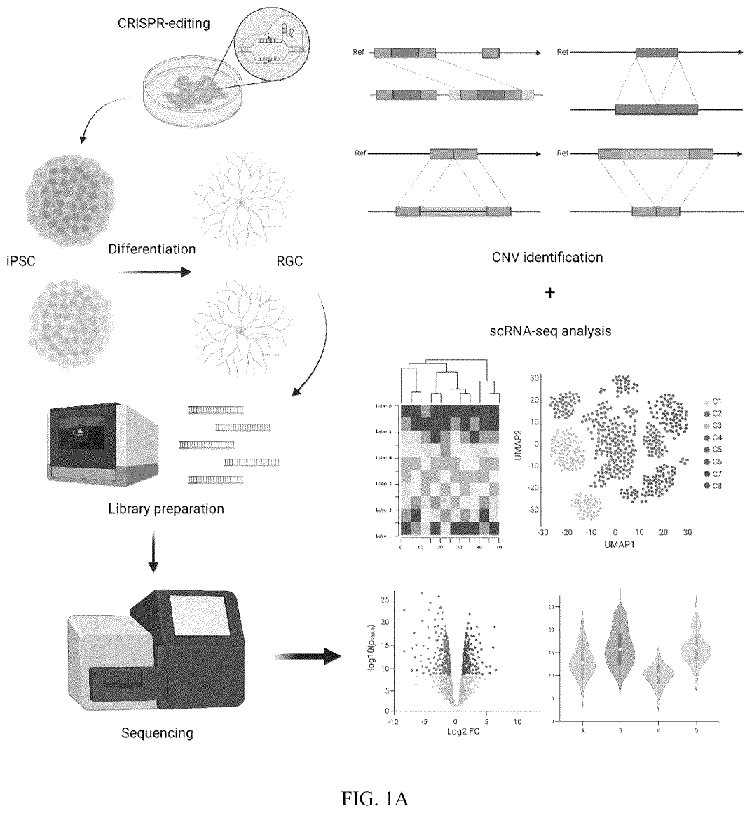

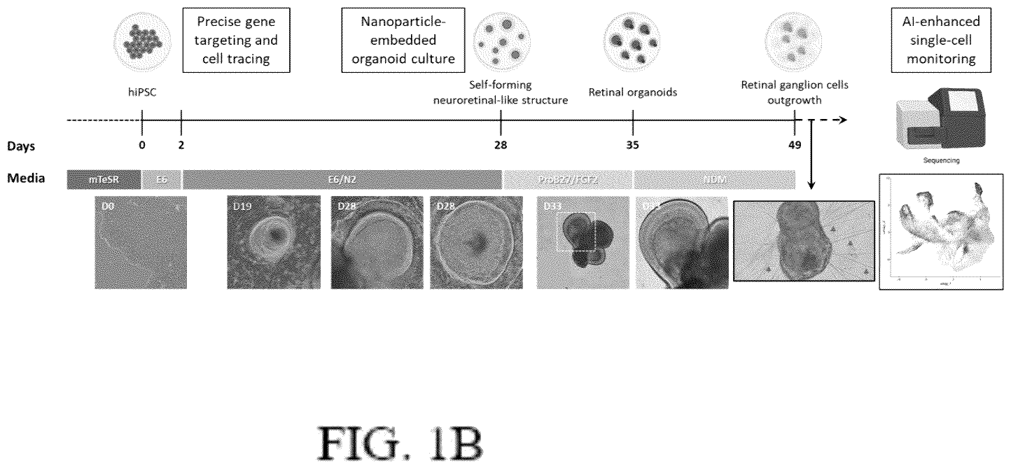

### DETAILED DESCRIPTION:

In the following description, methods of facilitating the differentiation of induced pluripotent stem cells (iPSCs) into retinal ganglion cells (RGCs) using CRISPR editing and the likes are set forth as preferred examples. It will be apparent to those skilled in the art that modifications, including additions and/or substitutions may be made without departing from the scope and spirit of the invention. Specific details may be omitted so as not to obscure the invention; however, the disclosure is written to enable one skilled in the art to practice the teachings herein without undue experimentation.

In accordance with a first aspect of the present invention, a comprehensive method for facilitating the differentiation of iPSCs into RGCs through a stepwise and targeted approach is provided. The method begins with the removal of a sequence encoding a long non-coding RNA, LNC000093, from the genome of iPSCs.

Removal of this sequence facilitates differentiation into RGCs. In one aspect, a CRISPR gene manipulation technique may be used to knockout the LNC000093 coding sequence (SEQ ID: 03: LNC000093 gene sequence: CTTGAAACAAAATACTGGTAAAGAAAAAGCTATCTATTCTAGTGGATA GAAATCTGGAGAAATATCACAAATGTTCTGAGCCCCAGACCTACTGAG ACTTTTGCAGGAAGCATTACCTCTATCCTACCTGGCAGACAAAACACTG CCTGCCCTTTTTTGCTAGATAATTCTAAGTGAGGAAGCCTGTGGACTTA CTAAATGACCTATATGATCTGCGAGGAGTAAAACACAACCATTCTGAA GGGAAACATACCCGGAACCATAAGAGTCTTTCACTAGGACCAGGGTAA GATGGTTTGGGGGGAACTGTCAAATATTTATGGCTTTTTGTTCTTACAC TGAATAAGTAGAATTTTTAAAAAATGTCTGGTGGTGAAAAGAACTTAA AACAGTTGAAGTATTCGGGGTGTCTAAAGTAAATTAAAACAGGCTGGG TGCATCATAATTTAGTTAGCCAGTTCTTTATAGATAGACATTTAGTTTAT TTCTAGCTCTTTTCTATTTTTTGGCATGCAAGCATCATTGTACATGTATC TCATCACATTTATTTGATTATACTCTTATGATCATTGCCTTAAAATATAG TTACTAAGAGTTTGTAAAACTTCCAAACTTTTGATACATATTGTTAAGT TGCCCTCTAAAAATATAATAAAAGTTGCCATTTCCACTAAAAGTATATG AGAATAGTTGTTTTCTTAATACTGTCACCAATATTATGTATTACCATTTA TTAAAAATATTTTTTAAAACTTGTCTAATCTGAAATGTGAAAAATGGTA CATTGTTTTAAGATTACTTGTGAGAACAATGATCATAATCATTATCATT TTTTGCGGGAGTATCTTACTTTCCTCTCAAAATAGTTCAATGAGTAGGT CCTATTAATTCATATATTCATTGGCTATTTCTGCCTTCTAAAAATTAATT TGTGTGTATGTCTTTTGTGTGTTTTTCTATTAGGATTTTTGTTTTTTTATT TTTATTTTTATTTTTATTTTTTTTTGAGACGGAGTCTCGCTCTGTCGCTCA GGCTGGAGTGCAGTGGCGCGATCTCGGCTCACTGCAAGCTCCGCCTCC CGGGTTCACACCATTCTCCTGCCTCAGCCTCCTGAGTAGCTGGGACTAC AGGTGCCCACCACCACGCCTGGCTAATTTTTTGTATTTTTAGTAGAGAC GGGGTTTCACGGTGTTAGCCAGGATGGTCTCGATCTCCTGAACTCGTGA TCCGCCTGCCTTGGCCTCCCAAAGTGCTGGGATTACAGGCGTCAGCCAC CGCGCCCGGACTCTATTAGTATTTTTTTTTTTTTAGTGTAGAAGTGCTCC TTAAAGATTAAGGAAATCAAGTTTCTCCTGATAATCTTTTTTTGGGATG TGTCTTTAGCGGTACTTCTGTTGATCAAAAGTAACTGATAACTCAGAAA GTATGGAGAAAGTCCTAAAATTGAACACAAATAAAAACAAATGAACCT CAATGTGTATAAAAATGTTAACATAACCACACAGAATAAAAAAAATTA ATACAAGAGACTTTTGAATATATTACTTTGATGTACACTCTCAGGGAGA TACATTCCAAGGACAAGAAGTACTATGAAGAAATACTAAACATTACTT AGTAGGTTTCTTGTTAGTAGAAGTATTGAAGTATTGATAGAGTAATTTT GAAGTTATTTGCTCAATTTTAGAATGTTAGAATTGAGATAGATAGATAG GTTGATTGATATTGCTGGGAAAAAGACTCTTACTGAGGCATAAAGGTG GTACAAACATGAAATGGCACAAGGAAAGGAAGAACCCTTTGGTGTTTG ATTGGCACTGGAAAGTATGAACTTATAATTTTATATATATTGGATTTGT TTCTCTTAGCAACGTCTGGATTTGTAAACACGCTCCAACAAGAAGCAG GCTGCACAAGCTTTGTAGATCACAAGGAGCATTAACTCTCCCATGACCT TCAGATGTGGCCATAGGGGCTGATGGACAAAGAATTACCCCCTAGAGC TAGGAGCAATGGATACCAGGGAGTTTTCCATAGAAAGCTGAACCAGAG ACTTGCTCCATAACCAGGACAGGGATCTTCACAATCTCTGACCAGAAG GATTTGCTTACCACTGTGGAAACAATGACTGTCATGTTTCTTCTCTTCTT TTTTTGGTGCCAAATCTATCACCATTGGCGGAGAAAAAGAACCTTCTCT TAGACTGTAGGTGATCTGACCCCAGGAGCCACACTTGGAGATAACTAC CCATCACCAAGAGATCCTGGAATTTCAACCTGATGATATAACTGGATG GGACTTTGGGAGTATCTCCTGTGGGAAAAAGGAAAGAATTTCGTTTGG CAGGTCAAGTGTAGTTGGATATTTATGGGTGGCTAAAGGGGCAGTCTG TGGAAGAGACTTCTTGTTATTTCTTCCCAACATCCATTCCTTTCTTCTCC TATTATGAGAGATATTTTGAATTTTTGACAAATCCCAGTCTCCCTTGCA TCTAGATGTGGCCATGAGACTAAGTTTTGGCCAAAGGGGTATAAATGA TATTGTTGTACACAACTTTAGGCTTGTGACCTTAAAGTGAAACAGGTGT GCCCTACACTGCCCATTTTTTCGACCTTGTTTTAACTGTGGATGATGTGG ATGAGGTGGTGAGCTACTATGGACCATGAGAATCAGTGGAGCATACTA GAGATGGTGCAACAATAAGAGAAGGAGCCTGGGTCCCTGAATGACCTC ATGGGGTAGATGTCCTAAGCTAGTATCACACTATTTACTCCCAGACTGG TGGAGAAGAGAGAAATAATTTCTGGCGAATTCAAAACACTGTTATTTT GGAGCCCTGTTACATGCACCTTGACCTATATTCCAAATTAGATAAGTAA GATAGGTAAGATATCTTAATGCACTTTGGCATGCTATCATTCCCAAATA CATGGGAATTAAATTTTTTTGGTATAAATTTACACTGAGAAACAAAAGT ATCTAAAACTAAAAGTATTATTT). A gene “knockout” using CRISPR (Clustered Regularly Interspaced Short Palindromic Repeats) involves inactivating or completely removing a specific gene in an organism's genome.

In this knockout process, a short RNA sequence, called the guide RNA, is designed to match the target gene's DNA sequence. The gRNA directs the CRISPR-associated protein 9 (Cas9) enzyme to the precise location in the genome.

Cas9 is a nuclease (a type of enzyme) that can cut DNA. When the gRNA binds to its complementary DNA sequence in the genome, Cas9 makes a double-strand break (DSB) at that location. The cell attempts to repair the DSB using one of two primary mechanisms:

Non-Homologous End Joining (NHEJ):

This repair mechanism is error-prone and often introduces insertions or deletions (indels) at the break site. These indels can disrupt the gene by causing a frameshift mutation, rendering it nonfunctional.

Homology-Directed Repair (HDR):

In knockouts, HDR may introduce a predefined mutation to disable the gene.

The disruption caused by NHEJ or the intentional mutation introduced via HDR prevents the gene from being transcribed into functional RNA or translated into a functional protein.

Following the deletion/knockout, the LNC000093-knocked-out iPSCs are cultured to form iPSC colonies under defined conditions, ensuring optimal growth and stability.

The iPSC colonies are then dissociated to form embryoid bodies, which represent an early stage of differentiation. These embryoid bodies are cultured in a hES medium devoid of bFGF, facilitating their development. Once formed, the embryoid bodies are transferred to a gelatin-coated first plate supplemented with 10% FBS to enhance the formation of neural rosettes. Neural rosettes, indicative of early neural development, are subsequently detached from the gelatin-coated first plate and cultured in suspension. The suspended neural rosettes are maintained in hES medium containing 10% FBS and 10 μM Notch inhibitor DAPT for 5 to 7 days, leading to the formation of neurospheres.

The neurospheres are then transferred to a second plate to facilitate their differentiation into RGCs. The medium used on the laminin-coated plate is supplemented with fresh Notch inhibitor DAPT, replenished every two days, to support and sustain the differentiation process. The resulting RGCs exhibit characteristic features of mature retinal ganglion cells, including dendritic arbors and axonal projections.

In some embodiment, the second plate is a laminin-coated plate.

To ensure the validity and quality of the differentiated RGCs, the method includes validation steps such as the assessment of RGC-specific markers. The expression of markers including POU4F2, SNCG, and BRN3A is confirmed using RT-qPCR or immunostaining techniques. Additionally, the transcriptional profiles of intermediate and mature RGCs are monitored through single-cell RNA sequencing, providing detailed insights into the differentiation process and verifying the developmental trajectory of the cells.

The method further includes an optional step for enriching the differentiated RGC population. This is achieved through MACS, utilizing specific RGC markers to isolate a pure population of functional RGCs. This enrichment step ensures that the resulting cell population is highly specific and suitable for downstream applications, such as disease modeling or therapeutic interventions.

Overall, the described method provides a robust and reproducible protocol for generating high-quality retinal ganglion cells from iPSCs. It leverages advanced genome editing, precise culture conditions, and rigorous validation techniques to overcome existing challenges in RGC differentiation, ensuring efficiency, accuracy, and applicability for research and clinical purposes.

In accordance with a second aspect of the present invention, a method for facilitating the differentiation of iPSCs, specifically the LNC000093-knocked-out iPSCs, into retinal organoids is also introduced. This method involves a series of culturing steps, designed to optimize the differentiation process and support the formation of fully developed retinal organoids.

The method begins with culturing LNC000093-knocked-out iPSCs in Essential 6 medium for an initial period of 28 to 35 days. During this stage, the iPSCs develop into neuroepithelial-like structures, which serve as the foundation for subsequent organoid formation. To further enhance differentiation efficiency, the Essential 6 medium is replaced with Essential 6 medium supplemented with 1% N2 supplement after the first 2 days of culturing. This adjustment provides an enriched environment that promotes the efficient differentiation of iPSCs into neuroepithelial-like structures.

Essential 6 Culture Medium:

Essential 6 (E6) Medium is a defined, serum-free, and xeno-free culture medium designed primarily for maintaining human pluripotent stem cells (hPSCs), such as embryonic stem cells (ESCs) and induced pluripotent stem cells (iPSCs), under feeder-free conditions. It is formulated to provide only the critical components required for the maintenance of stem cell pluripotency and viability while eliminating other non-essential factors.

Composition of Essential 6 Medium

The medium includes six key components, which give it its name:

Basal Medium:

Often based on DMEM/F12 (Dulbecco's Modified Eagle Medium and Ham's F-12 Nutrient Mixture), providing essential nutrients, salts, and buffering capacity.

Insulin:

Aids in cell metabolism and survival by supporting glucose uptake and energy production.

Transferrin:

A key iron carrier protein that supports cell growth and prevents oxidative stress.

L-Ascorbic Acid (Vitamin C):

Promotes cell viability, protects against oxidative damage, and supports collagen synthesis.

Selenium:

A trace element essential for antioxidant defense and cell health.

Sodium Bicarbonate:

Helps maintain the pH balance in the culture medium.

Following the formation of neuroepithelial-like structures, these structures are transferred to a suspension culture medium for another 5-7 days. The suspension culture medium consists of DMEM/F12 medium supplemented with 1% MEM nonessential amino acids, 2% B27 supplement, and 10 ng/ml FGF2, which together support the growth and maturation of floating organoids. During this phase, the neuroepithelial-like structures gradually develop into well-formed floating organoids.

Once the floating organoids have matured, they are plated onto a Matrigel-coated surface and cultured for an additional 5 to 7 days. This step promotes the final stages of retinal organoid development, enabling the formation of structurally and functionally competent organoids. Notably, the Matrigel-coated surface may be pre-treated with selenium nanoparticles (SeNPs) to further enhance the development and function of the retinal organoids. The SeNPs, introduced at a concentration of approximately 0.5-1 μM into the culture medium before harvesting, provide additional support for organoid development by enhancing structural integrity and promoting functional properties.

Finally, the retinal organoids are harvested, completing the differentiation process. The method highlights the critical role of SeNP treatment in enhancing the overall quality and functionality of the retinal organoids, as evidenced by improved development and function.

This comprehensive approach provides a robust protocol for generating retinal organoids with high differentiation efficiency and functional capabilities, making it suitable for applications in disease modeling, drug screening, and potential therapeutic interventions.

### EXAMPLES

### Example 1. CRISPR-Cas9-Mediated Deletion of Target Gene

A long non-coding RNA named LNC000093 is selected as a target gene for knockout to investigate its effect on iPSC differentiation towards RGCs. The Alt-R CRISPR-Cas9 system (IDT) is used to generate LNC000093-deletion in iPSCs. A pair of custom sgRNAs targeting two genomic regions flanking LNC000093 is designed using proprietary algorithms provided by IDT. The sgRNAs are mixed with Alt-R® S.p. Cas9 Nuclease V3 (IDT) to form ribonucleoprotein (RNP) complexes, which are subsequently used to assemble transfection complexes by incubation with Lipofectamine RNAiMAX reagent (Invitrogen). A total of 3×10⁵ cells per well in 24-well plates are transfected with 10 nM RNP complex.

A pair of PCR primers, including a forward primer (SEQ ID NO: 01: 5′-ATG TTG GTG TAT CTT GAG ATC CTC-3′; and a reverse primer (SEQ ID NO: 02: 5′-TCC CCA GTT GTA CTC CAT CTG T-3′, is designed to target the regions flanking the deletion region of LNC000093. Genomic DNA is extracted from 1×10⁶ CRISPR-edited cells using the FlexiGene DNA Kit (Qiagen) according to the manufacturer's instructions. A total of 60 ng DNA is employed in each PCR reaction using Platinum SuperFi II Green PCR Master Mix (Invitrogen) under the following cycling conditions: 98° C. for 30 s; 30 cycles of 98° C. for 15 s, 60° C. for 15 s, 72° C. for 2.5 min; and 72° C. for 5 min. The PCR amplicons are analyzed by 2% agarose gel electrophoresis to assess the CRISPR-deletion effect based on the size difference of the amplified product.

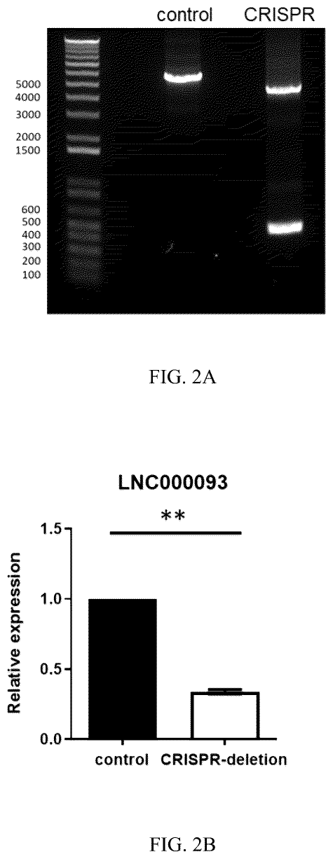

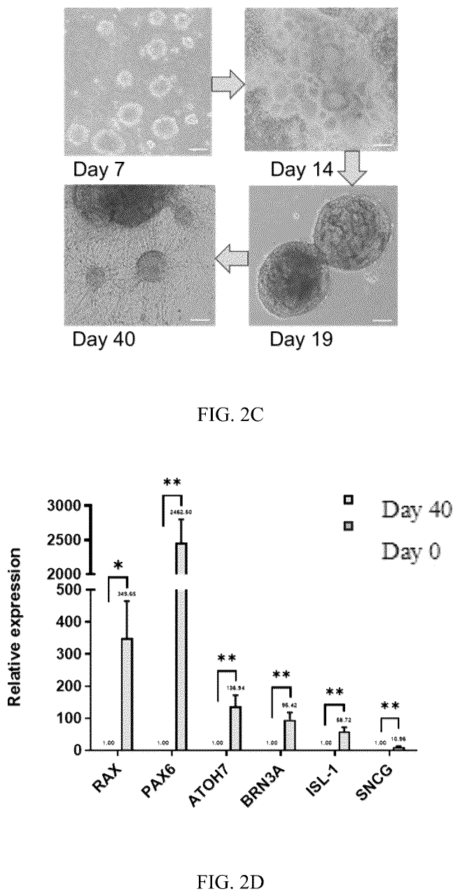

The gene editing effect of LNC000093 CRISPR-deletion in iPSCs is evaluated using PCR assays. Gel electrophoresis results for the LNC000093-CRISPR sample show two bands (FIG. 2A). The smaller amplicon represents cells with LNC000093 deletion since the gene region is excised, resulting in a shorter DNA segment for amplification, thereby confirming the CRISPR-mediated deletion. Furthermore, RNA is extracted for RT-qPCR analysis to assess the RNA expression level of LNC000093 after CRISPR-deletion. The qPCR results demonstrate a significant downregulation of LNC000093 in the CRISPR-deleted sample compared to the control, validating the effectiveness of the gene knockout (FIG. 2B).

### Example 2. iPSC-RGC Differentiation

iPSCs are differentiated towards RGCs in a stepwise manner using specified culture media containing cytokines and small molecules at different stages. Embryoid bodies (EBs) are formed by dissociating iPSC colonies into small aggregates and growing them in suspension for 7 days in human embryonic stem (hES) medium without basic fibroblast growth factor (bFGF). The EBs are subsequently transferred to gelatin-coated plates and cultured in hES medium supplemented with 10% fetal bovine serum (FBS) to induce the formation of neural rosettes. After one week, the neural rosettes are carefully detached using a syringe needle and a pipette tip, after which they are cultured in suspension in hES medium containing 10% FBS and 10 μM Notch inhibitor DAPT for five days to form neurospheres.

The neurospheres are transferred to plates coated with laminin, and the culture medium, along with fresh DAPT, is replenished every other day. By day 40, differentiated cells are harvested using Accutase treatment for subsequent experiments. iPSCs progress through different stages of differentiation (FIG. 2C). By day 7, the emergence of EBs marks the initial stages of differentiation. By day 14, mature neural rosettes become apparent, representing early neural development and retinal differentiation. These structures are further cultured into neurospheres by day 19, which differentiate towards the RGC lineage over 21 days.

By day 40, RGCs with characteristic dendritic arbors and axonal projections are observed and collected for subsequent experiments. RT-qPCR analysis is conducted to validate the cell identity of RGCs, targeting well-established RGC-specific markers, including RAX, PAX6, ATOH7, BRN3A, ISL1, and SNCG. Compared to undifferentiated iPSCs (day 0), the expression of all six marker genes is significantly upregulated in iPSC-derived RGCs, affirming the successful differentiation (FIG. 2D).

### Example 3. iPSC-Retinal Organoid Differentiation

To initiate retinal organoid differentiation, iPSCs are grown to 70-80% confluence before transitioning to Essential 6 medium on day 0 of differentiation. After two days, the culture medium is replaced with E6N2 medium (Essential 6 medium supplemented with 1% N2 supplement), and the medium is changed every 2-3 days. By day 28, neuroepithelial-like structures self-form in adherent culture and are manually isolated using a syringe needle. These structures are transferred to floating cultures in PRO medium (DMEM/F12 supplemented with 1% MEM nonessential amino acids, 2% B27 supplement, and 10 ng/ml FGF2). Floating organoids are maintained with half-medium changes every 2-3 days.

On day 35, the floating organoids are plated onto Matrigel-coated surfaces and cultured for an additional week in NDM medium (Neurobasal medium supplemented with 1% MEM non-essential amino acids, 1% GlutaMAX, 1% glucose (45%), 2% B27, and 1% N2). Medium changes are performed every 2-3 days during this stage. Selenium nanoparticle (SeNP) treatment is introduced by premixing 1 μM SeNP-LBP with the Matrigel coating on day 35 or by directly adding 1 μM SeNP-LBP into the culture medium on day 41. Organoids are harvested on day 42, dissociated with Accutase, and gently pipetted every 10 minutes during a 30-minute incubation at 37° C. This process yields retinal organoids suitable for downstream analyses.

### Example 4. Single-Cell RNA Sequencing Analysis of RGCs

Day 40 cell samples are collected as described in the previous section and subjected to single-cell RNA sequencing (scRNA-seq) library preparation using 10X Genomics technology. The preparation employs the Chromium Next GEM Single Cell 3′ Reagent Kit v3.1 (PN-1000269), the Chromium Next GEM Chip G Single Cell Kit (PN-1000127), and the corresponding user guide (document code CG000315_Rev E). Cell suspensions are loaded onto a microfluidic chip and processed in the Chromium Controller to generate Gel Beads-in-Emulsion (GEMs), encapsulating single nuclei. The GEMs produce 10X-barcoded, full-length cDNA from RNA after incubation. All steps to generate Single Cell 3′ Gene Expression libraries are conducted per the manufacturer's protocol. The quality and quantity of the cDNA and gene expression library are assessed using an Agilent 2100 Bioanalyzer with the High Sensitivity DNA kit. Sequencing is performed on an Illumina NovaSeq platform.

The raw sequencing data in bel files are converted to FASTQ files using CellRanger (version 7.1). These FASTQ files are aligned to the human genome reference sequence GRCh38 using default settings. CellRanger generates output folders for each sample containing a barcode table, a gene table, and a gene expression matrix for downstream analysis.

Doublets are identified and excluded using Scrublet (version 0.2.3), based on a recommended 6% doublet rate from 10X guidelines. Data preprocessing and cell clustering are conducted using the Seurat package (version 4.4.0) in R (version 4.3.1). The scRNA-seq datasets are subjected to quality control, normalization, and feature selection, followed by integration using the anchor method in Seurat. The integration process identifies anchors—cell pairs in similar biological states from different samples—via canonical correlation analysis (CCA). Once integrated, the dataset undergoes data scaling, dimensional reduction via principal component analysis (PCA), clustering, Uniform Manifold Approximation and Projection (UMAP) reduction, and visualization of clusters.

Marker genes indicative of differentiation paths are identified to distinguish retinal progenitor cells and mature RGCs. RNA velocity analysis, conducted using the velocyto package (version 0.17), anticipates the developmental trajectory. RNA velocity evaluates the ratio of unspliced to spliced RNA transcripts to infer transcriptional dynamics, providing insights into cell transitions and differentiation processes. Percentages of cells in each cluster are calculated and statistically compared between control and LNC000093-CRISPR samples using Student's t-test.

To assess the impact of LNC000093 deletion on gene expression, differentially expressed genes (DEGs) are identified in RGC-specific clusters using the FindMarkers function in Seurat. Genes with |log2 fold change|>2 and adjusted p-value<0.05 are considered DEGs. Significant DEGs undergo Gene Ontology (GO) enrichment analysis using the ClusterProfiler package (version 4.10.0).

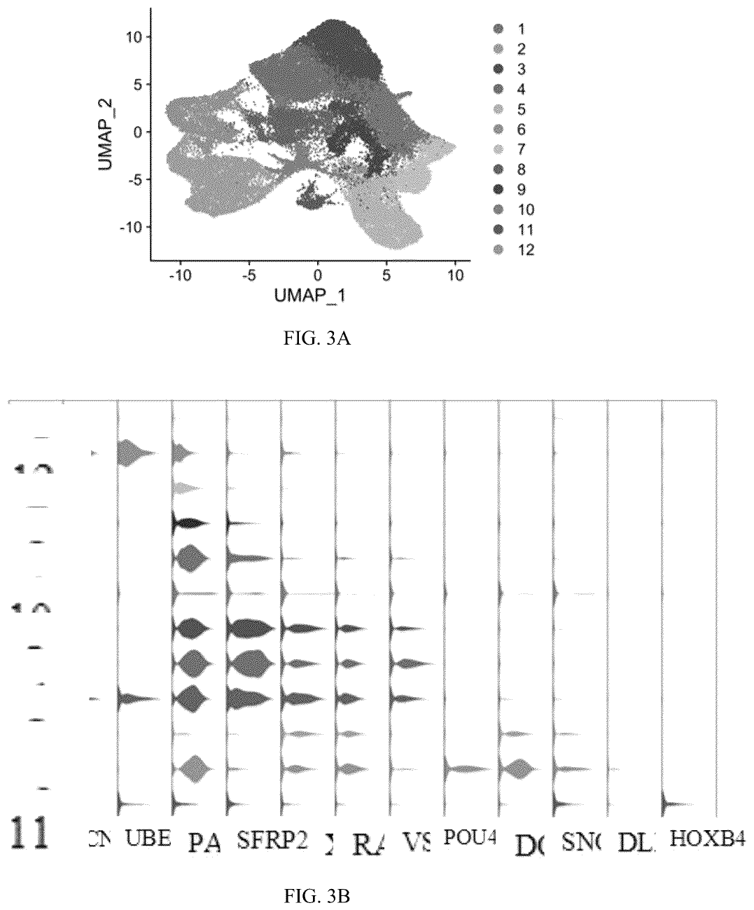

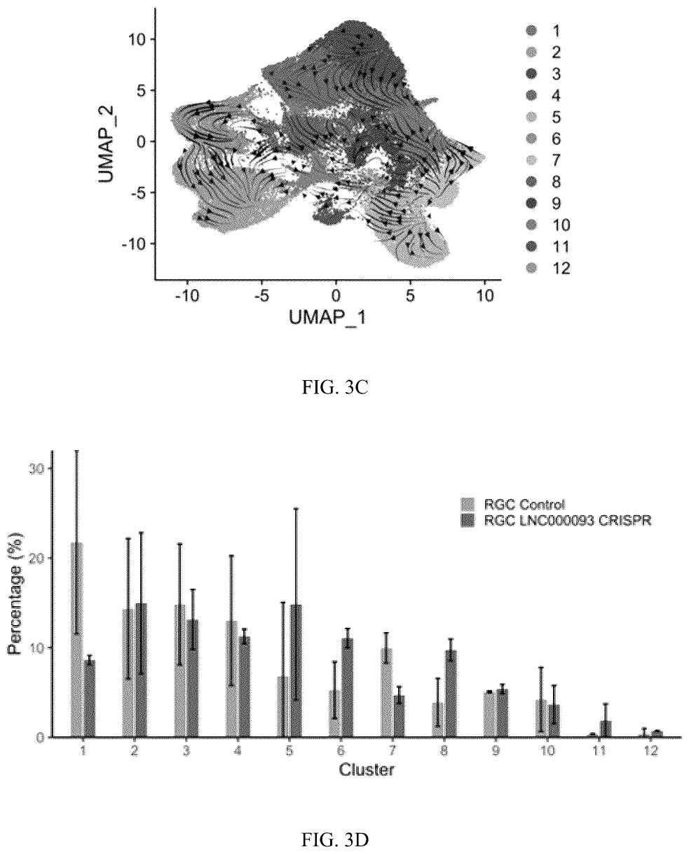

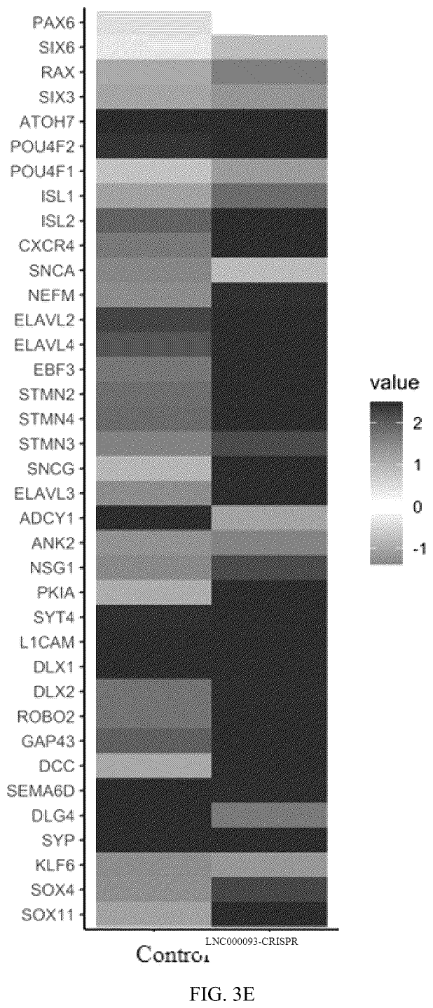

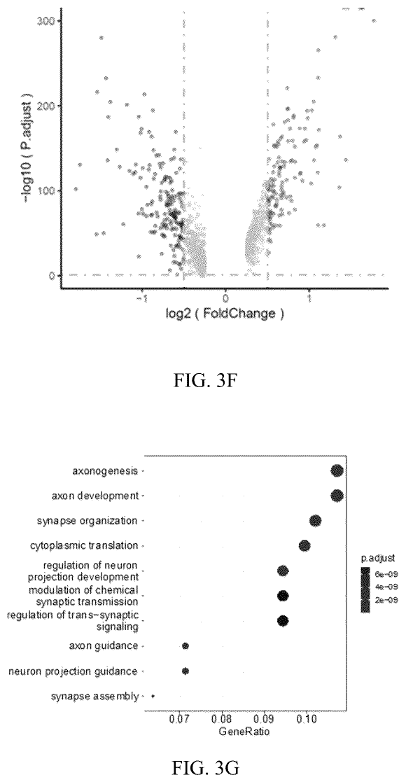

Initial doublet analysis excludes approximately 200 doublets per sample. Subsequent clustering analysis identifies 12 distinct cell clusters (FIG. 3A). Based on RGC marker expression, cluster 2 is annotated as the immature RGC cluster, and cluster 6 as the mature RGC cluster (FIG. 3B). Clusters 1, 3, 4, 5, 7, 8, 9, 10, and 12 are identified as progenitor clusters, with high expression of retinal progenitor markers PAX6, SFRP2, and RAX. Cluster 11 deviates, representing a distinct path toward non-retinal neurons based on the neuron gene marker HOXB4 (FIG. 3B). RNA velocity analysis supports differentiation paths from clusters 5 and 7 to clusters 2 and 6 (FIG. 3C).

Comparison between control and LNC000093-CRISPR samples reveals a significant increase in cluster 6 (mature RGCs) for the LNC000093-CRISPR sample (FIG. 3D). Further analysis of RGC-specific marker genes and axon guidance-related genes indicates higher expression levels in the LNC000093-CRISPR sample, suggesting enhanced maturation of RGCs (FIG. 3E).

Within clusters 2 and 6, DEGs are identified between conditions, yielding 471 upregulated genes and 163 downregulated genes (FIG. 3F). GO analysis of the upregulated genes reveals significant enrichment in axonogenesis and axon development (FIG. 3G), suggesting enhanced axonal functions in the LNC000093-CRISPR sample. These findings underscore the potential role of LNC000093 deletion in promoting RGC maturation and axonogenesis.

### Example 5. Single-Cell RNA Sequencing Analysis of Retinal Organoids

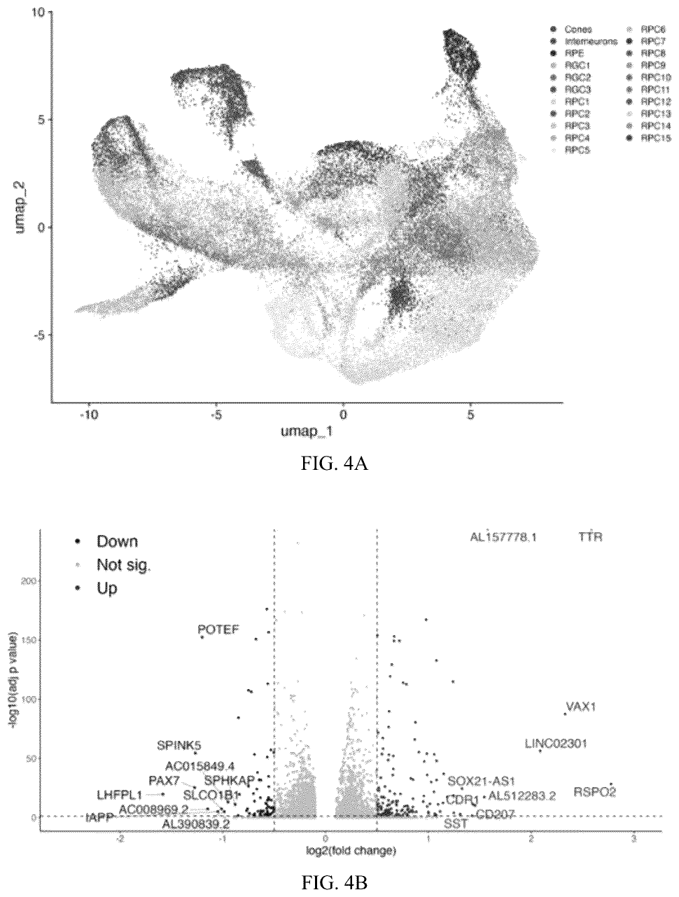

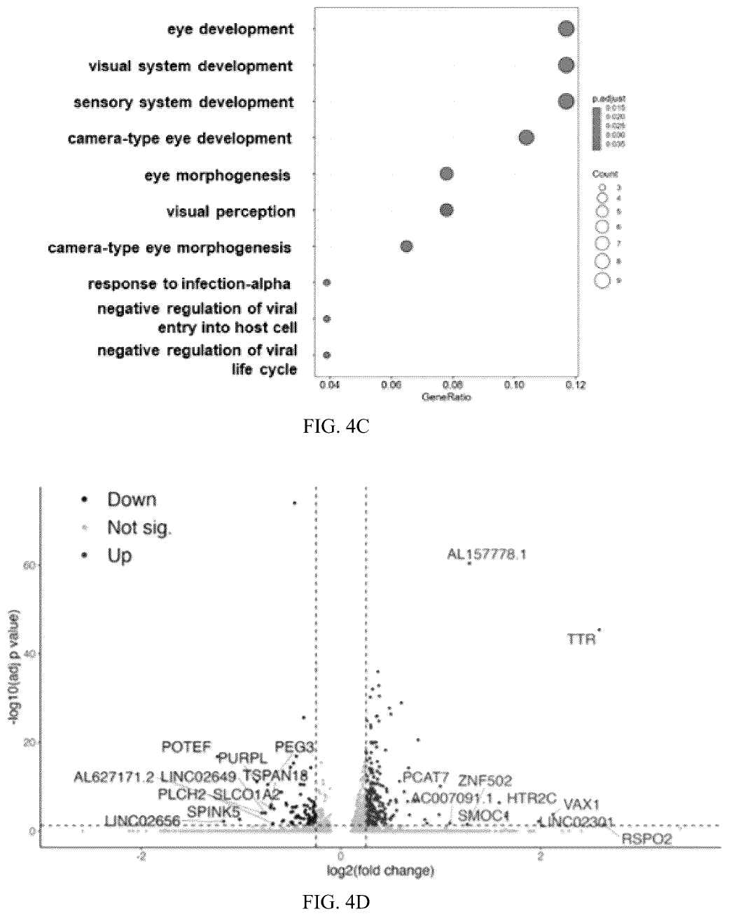

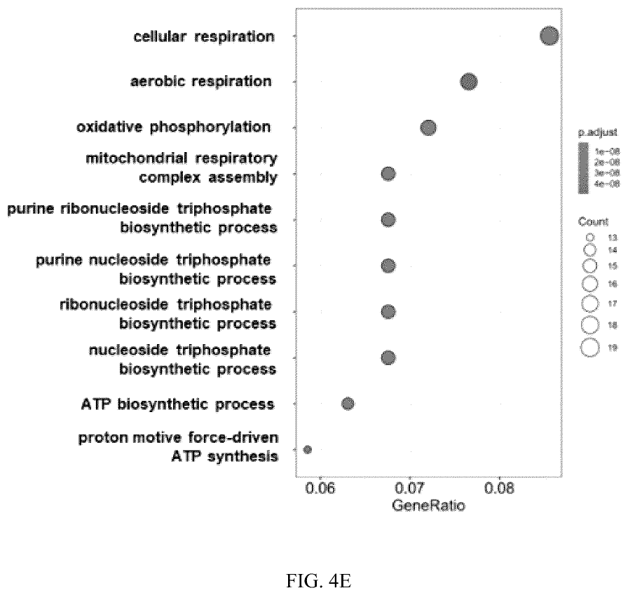

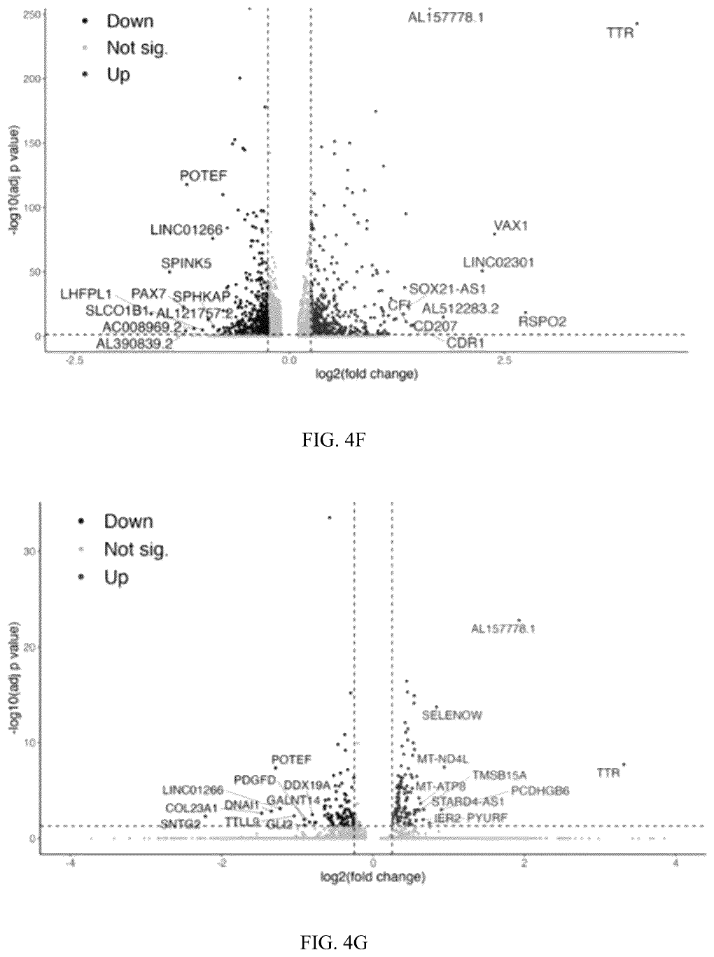

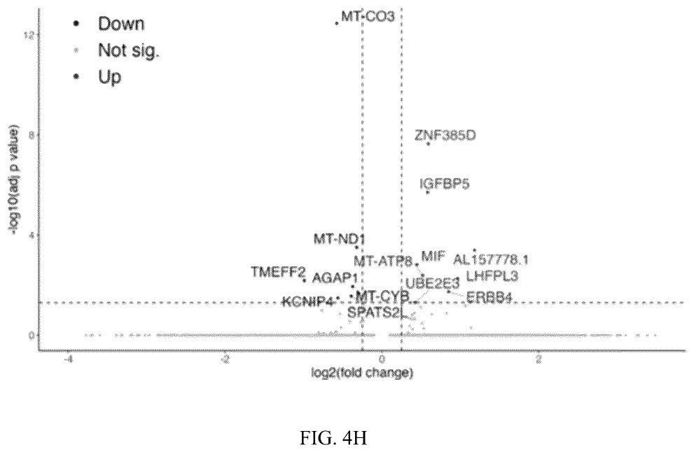

A total of 42,983 cells are initially obtained from four retinal organoid samples. After filtering out low-quality cells based on mitochondrial counts and unique feature counts, 38,909 high-quality cells are retained for downstream analysis. Clustering analysis identifies 21 distinct cell clusters representing major retinal cell types, including cones, retinal ganglion cells (RGCs) (RGC1-RGC3), retinal pigment epithelium (RPE), retinal progenitor cells (RPCs) (RPC1-RPC15), and interneurons (FIG. 4A). Differentially expressed gene (DEG) analysis identifies 154 upregulated and 76 downregulated genes in the selenium-nanoparticle-based Lycium barbarum polysaccharide (SeNP-LBP) condition compared to the control organoid (FIG. 4B). Gene Ontology (GO) enrichment analysis of the upregulated genes reveals significant enrichment in biological processes related to eye development, visual system development, and sensory system development (FIG. 4C), suggesting that SeNP-LBP treatment induces transcriptional changes that potentially enhance retinal organoid development and function.

To explore cell type-specific transcriptional changes, DEGs are analyzed for each major retinal cell type. In RGCs, 262 upregulated and 77 downregulated genes are identified in the SeNP-LBP group compared to the control (FIG. 4D). GO enrichment analysis highlights biological processes related to energy metabolism and oxidative phosphorylation (FIG. 4E), indicating a potential role of SeNP-LBP in regulating mitochondrial activity in RGCs. For RPCs, DEG analysis identifies 527 upregulated and 436 downregulated genes, while cones exhibit 112 upregulated and 101 downregulated genes (FIG. 4F, FIG. 4G). The RPE shows a smaller transcriptional response with eight upregulated and seven downregulated genes (FIG. 4H). These results indicate that SeNP-LBP induces cell-type-specific transcriptional responses, with notable effects on RGCs and RPCs.

### Example 6. Genome Integrity Confirmation Via the CNVPipe Bioinformatics Pipeline

Genomic DNA from control and LNC000093-CRISPR iPSCs is subjected to whole genome sequencing (WGS) using the Illumina NovaSeq platform. Raw data quality is assessed using FastQC (version 0.12.0), and base quality information is extracted. Data processing, including trimming, mapping, and CNV calling, is performed using the CNVPipe pipeline with default settings.

In the CNVPipe workflow, fastp is used to remove adaptors and low-quality reads, while GATK4 recalibrates base quality and identifies duplicate reads, resulting in bam files. CNVPipe then incorporates five widely recognized CNV-calling tools—CNVKit, CNVpytor, cn.MOPS, Delly, and Smoove—and employs a merging strategy based on sequencing depth. CNVs with a reciprocal overlap larger than 0.75 are merged, and new CNV boundaries are determined using the outermost breakpoints.

For comparing CNVs between samples, the Bedtools intersect function (version 2.28.0) identifies common CNVs with a reciprocal overlap exceeding 0.25 and matching CNV types (deletion or duplication). Remaining unique CNVs are cross-referenced with the human genome reference (hg38) to determine whether they overlap with known gene regions, enabling the identification of potential off-target effects from CRISPR editing.

Using 30x whole-genome sequencing (WGS) data from both iPSC control and CRISPR-edited samples, CNVPipe determines a resolution of 300 base pairs (bp) for detecting copy number variations (CNVs). The pipeline integrates five widely used CNV-calling tools—CNVKit, CNVpytor, cn.MOPS, Delly, and Smoove—to independently identify CNVs. The outputs from these tools are merged to create a unified CNV set. To ensure the quality and accuracy of the identified CNVs, scoring metrics are calculated for each CNV. Additionally, a support vector machine (SVM) classifier is trained using these scoring metrics along with genomic features such as copy number values, GC content, and depth ratio relative to adjacent genomic regions. This trained classifier predicts true positive CNVs within the merged CNV set. The final CNV set is refined by visually examining read depth plots in CNV regions and their adjacent areas for further validation.

With this methodology, CNVPipe identifies 238 CNVs in the iPSC control sample and 230 CNVs in the LNC000093-deleted sample. After filtering out common CNVs shared between the two samples, 16 unique CNVs are identified in the control iPSC sample and 22 unique CNVs in the CRISPR-edited sample (Table 1). These unique CNVs are further annotated to determine their overlap with known gene regions. Importantly, none of the unique CNVs in the edited sample overlaps with any known genes, confirming the absence of off-target effects from the CRISPR-mediated deletion of LNC000093.

TABLE 1
Unique CNVs identified in control and CRISPR-edited iPSC
samples using CNVPipe.

| Unique CNVs from control iPSC | | | | | Unique CNVs from LNC000093-CRISPR iPSC | | | | |
|---|---|---|---|---|---|---|---|---|---|
| chr | start | end | type | gene | chr | start | end | type | gene |
| chr2 | 95497544 | 95659739 | duplication | NA | chr1 | 13152287 | 13223987 | deletion | NA |
| chr2 | 169785008 | 169787472 | deletion | NA | chr1 | 148621197 | 148634096 | deletion | NA |
| chr3 | 68584694 | 68591327 | deletion | NA | chr1 | 228067627 | 228072406 | deletion | NA |
| chr5 | 50938436 | 50943629 | deletion | NA | chr2 | 34470762 | 34511499 | deletion | NA |
| chr6 | 103289401 | 103315346 | deletion | NA | chr2 | 106262639 | 106269599 | deletion | NA |
| chr7 | 23998339 | 24000491 | deletion | NA | chr2 | 193824641 | 193834040 | deletion | NA |
| chr8 | 74450512 | 74454790 | deletion | NA | chr3 | 68690531 | 68698799 | deletion | NA |
| chr10 | 79842567 | 79851866 | deletion | NA | chr4 | 186407057 | 186609518 | duplication | NA |
| chr11 | 55666201 | 55692223 | deletion | NA | chr5 | 143481502 | 143486699 | deletion | NA |
| chr12 | 8405877 | 8438576 | deletion | NA | chr6 | 76387779 | 76392923 | deletion | NA |
| chr14 | 20081829 | 20087399 | deletion | NA | chr7 | 126411372 | 129230868 | deletion | NA |
| chr15 | 28357365 | 28370563 | deletion | NA | chr8 | 12229844 | 12323043 | deletion | NA |
| chr16 | 35228701 | 35524036 | duplication | NA | chr9 | 39025160 | 39079159 | duplication | NA |
| chr17 | 21642550 | 21770049 | duplication | NA | chr9 | 41957365 | 41969399 | duplication | NA |
| chr18 | 41284738 | 41288399 | deletion | NA | chr10 | 47480367 | 47552366 | deletion | NA |
| chr18 | 44396679 | 44402108 | deletion | NA | chr10 | 49795201 | 49803007 | deletion | NA |
| | | | | | chr10 | 65546814 | 65555999 | deletion | NA |
| | | | | | chr11 | 124250373 | 124251469 | deletion | NA |
| | | | | | chr13 | 50495161 | 50501116 | deletion | NA |
| | | | | | chr16 | 25328642 | 25331805 | deletion | NA |

The workflow greatly enhances the improvement of iPSC-RGC differentiation by incorporating CRISPR editing, single-cell RNA sequencing analysis, and CNV detection using CNVPipe.

In conclusion, the method of the present invention introduces an integrated and revolutionary workflow for enhancing the differentiation of iPSCs into RGCs. This comprehensive approach combines synthetic RNA-based CRISPR editing, single-cell RNA sequencing analysis, and the CNVPipe bioinformatics pipeline for genome integrity confirmation, addressing critical challenges in the field.

The foremost innovation lies in the use of a non-viral, synthetic RNA-based CRISPR editing system for precise genome modification, exemplified by the targeted removal of LNC000093 to promote iPSC-RGC differentiation. This strategy eliminates the inherent risks associated with viral vectors, such as off-target effects and immunogenicity, providing a safer and more efficient method for gene editing.

A key feature of this invention is its incorporation of single-cell RNA sequencing, which allows for real-time monitoring of iPSC differentiation and ensures a detailed understanding of cellular transitions. This step addresses the limitations of existing methods that often lack precision, resulting in heterogeneous cell populations.

Furthermore, the CNVPipe bioinformatics pipeline offers a cutting-edge solution for evaluating genome integrity post-CRISPR modification. Unlike conventional tools, CNVPipe introduces advanced algorithms specifically designed for accurate CNV detection, ensuring a meticulous and reliable assessment of genomic stability.

By seamlessly integrating these elements into a unified workflow, the present invention significantly enhances the efficiency, accuracy, and safety of RGC differentiation from iPSCs. This comprehensive toolkit revolutionizes the field of regenerative medicine, providing researchers with a robust framework for precise genome editing and reliable evaluation of cellular outcomes. 5

The foregoing description of the present invention has been provided for the purposes of illustration and description. It is not intended to be exhaustive or to limit the invention to the precise forms disclosed. Many modifications and variations will be apparent to the practitioner skilled in the art.

The embodiments were chosen and described in order to best explain the principles of the invention and its practical application, thereby enabling others skilled in the art to understand the invention for various embodiments and with various modifications that are suited to the particular use contemplated.

1. A method for facilitating the differentiation of induced pluripotent stem cells (iPSCs) into retinal ganglion cells (RGCs), comprising:
deleting a sequence encoding a long non-coding RNA, LNC000093, from the genome of iPSCs, wherein the LNC000093 has a sequence of SEQ ID NO: 03;
culturing the LNC000093-knocked-out iPSCs to generate iPSC colonies;
dissociating the iPSC colonies to form embryoid bodies;
transferring the embryoid bodies to a first plate to induce the formation of neural rosettes on the gelatin-coated first plate;
detaching the neural rosettes from the first plate and culturing the detached neural rosettes in suspension to form neurospheres; and
transferring the neurospheres to a second plate to facilitate the differentiation into RGCs.

2. The method of claim 1, wherein the first plate is a gelatin-coated plate supplemented with 10% fetal bovine serum (FBS) to enhance neural rosette formation.

3. The method of claim 1, wherein the detached neural rosettes are cultured in suspension in hES medium comprising 10% FBS and 10 μM Notch inhibitor DAPT for a period of 5 days to form the neurospheres.

4. The method of claim 1, wherein the formation of the embryoid bodies comprises culturing the iPSC colonies in a human embryonic stem (hES) medium without basic fibroblast growth factor (bFGF).

5. The method of claim 1, further comprising maintaining the second plate with a medium supplemented with fresh Notch inhibitor DAPT, replenished every two days, to support RGC differentiation.

6. The method of claim 1, further comprising validating the identity of the retinal ganglion cells by assessing the expression of retinal ganglion cell-specific markers, comprising POU4F2, SNCG, and BRN3A, using RT-qPCR or immunostaining techniques.

7. The method of claim 1, further comprising monitoring the differentiation process through single-cell RNA sequencing to evaluate the transcriptional profiles of intermediate and mature RGCs.

8. The method of claim 1, further comprising enriching the resulting RGCs through magnetic-activated cell sorting (MACS) based on the expression of specific RGC markers.

9. The method of claim 1, wherein the second plate is a laminin-coated plate.

10. A method for facilitating retinal organoid differentiation, comprising:
culturing the LNC000093-knocked-out iPSCs of claim 1 in an Essential 6 medium for approximately 28 days to form neuroepithelial-like structures;
transferring the neuroepithelial-like structures to a suspension culture medium for approximately 7 days to form floating organoids;
plating the floating organoids onto a Matrigel-coated surface and culturing them for approximately 7 days to form retinal organoids; and
harvesting the retinal organoids.

11. The method of claim 10, wherein the Matrigel-coated surface is pre-treated with selenium-nanoparticles (SeNPs).

12. The method of claim 11, wherein the SeNPs are introduced into the culture medium at a concentration of approximately 1 μM prior to harvesting the retinal organoids.

13. The method of claim 10, wherein the Essential 6 medium is replaced with an Essential 6 medium supplemented with 1% N2 supplement after 2 days of initial culturing to enhance differentiation efficiency.

14. The method of claim 10, wherein the suspension culture medium comprises a DMEM/F12 medium supplemented with 1% MEM nonessential amino acids, 2% B27 supplement, and 10 ng/ml FGF2 to support the growth of floating organoids.

15. The method of claim 10, wherein the treatment of SeNP enhances the development and functions of the retinal organoids.
